(19) United States
(12) Patent Application Publication
(10) Pub. No.: US 2024/0423971 A1
(43) Pub. Date: Dec. 26, 2024

(54) APPLICATION OF RIP2 INHIBITOR COMBINED WITH CHEMOTHERAPY DRUG

(71) Applicant: NINGBO COMBIREG PHARMACEUTICAL TECHNOLOGY CO., LTD., Zhejiang (CN)

(72) Inventors: Gang LIU, Zhejiang (CN); Yao MA, Zhejiang (CN); Xiduan WEI, Zhejiang (CN)

(21) Appl. No.: 18/710,211

(22) PCT Filed: Nov. 17, 2022
(86) PCT No.: PCT/CN2022/132514

§ 371 Date: May 15, 2024

(30) Foreign Application Priority Data
Nov. 22, 2021 (CN) 202111388792.0

(51) Int. Cl. A61K 31/4706; A61K 31/282; A61K 31/337; A61K 31/7068; A61P 35/00

(52) U.S. Cl. CPC A61K 31/4706; A61K 31/282; A61K 31/337; A61K 31/7068; A61P 35/00

(57) ABSTRACT

An application of a receptor-interacting protein 2 (RIP2) inhibitor combined with a chemotherapy drug, particularly, an application of a RIP2 inhibitor combined with a chemotherapy drug in preparation of a drug for treating and/or preventing a tumor. For the first time, the RIP2 inhibitor is found to enhance the antitumor activity of chemotherapy drugs; after combined administration, the ratios of CD45+ cells, neutrophils, dendritic cells, macrophages, CD4+T cells, CD8+T cells, and γ/δ T cells in the tumor microenvironment are up-regulated, and tumor chemotherapy resistance is reversed.

The present application claims the right of the priority of Chinese patent application 2021113887920 filed on Nov. 22, 2021. The contents of the above Chinese patent application are incorporated herein by reference in their entireties.

### TECHNICAL FIELD

The present disclosure belongs to the technical field of tumor treatment and relates to a use of a RIP2 inhibitor combined with a chemotherapy drug; specifically, it relates to a use of a RIP2 inhibitor in enhancing the efficacy of antitumor chemotherapy drugs.

### BACKGROUND

Tumor chemotherapy is a conventional means of antitumor treatment. Chemotherapy after surgery (adjuvant chemotherapy) and before surgery (neoadjuvant chemotherapy) can improve the cure rate of some cancers and increase the chance of surgical resection of various solid tumors in locally advanced stages. However, the chemotherapy drugs currently used in clinical practice not only have long treatment cycles but also require high doses, leading to varying degrees of toxic side effects of various chemotherapy drugs (alkylating agents, antimetabolites, antitumor antibiotics, plant-derived antitumor drugs, platinum-based drugs, etc.) with different mechanisms of action, such as bone marrow suppression, gastrointestinal reactions, cardiotoxicity, pulmonary toxicity, hepatotoxicity, nephrotoxicity, bladder toxicity, neurotoxicity, etc., all of which severely affect the clinical use of chemotherapy drugs. Therefore, the search for a substance that can enhance the efficacy of chemotherapy, reduce its therapeutic dose, and thereby alleviate its toxic side effects has been a long-standing goal of scientists.

Pattern recognition receptors NOD1 and NOD2 are important components of innate immunity, and research involving NOD½ antagonists in the field of antitumor immunotherapy has made significant progress. For example, NOD1 receptor serves as a therapeutic target for inflammation-mediated colon cancer metastasis, and activation of the NOD1 signaling pathway can increase the adhesion, migration, and metastasis of colorectal cancer cells; NOD2 serves as an intestinal immune checkpoint, and deletion of NOD2 can inhibit intestinal bacterial translocation, thereby shortening the immune-dependent antitumor effects mediated by cyclophosphamide (CTX). Therefore, in the development of cancer treatment strategies, dual antagonists of NOD1 and NOD2 that effectively assist in cancer treatment should have a broader prospect. Receptor-interacting protein 2 (RIP2) is a key protein in the NOD½ downstream signaling pathway. The phosphorylation activation of RIP2 is considered to be a hallmark of the activation of the NOD½ signaling pathway. Targeting RIP2 is a feasible approach to achieve dual antagonism of the NOD1 and NOD2 signaling pathways. GSK583 is a highly efficient and highly selective small molecule compound inhibitor of RIP2 kinase, which can significantly inhibit the activation of the NOD½ downstream signaling pathway and the release of inflammatory cytokines such as IL8, IL6, and TNFα. Its role in antitumor treatment has not yet been reported, but it is a potentially high-quality small molecule for sensitizing antitumor chemotherapy drugs.

### CONTENT OF THE PRESENT INVENTION

In order to solve the above technical problems, the present disclosure provides a use of a RIP2 inhibitor combined with a chemotherapy drug. The purpose of the present disclosure is to provide a new use of a RIP2 inhibitor that can significantly sensitize antitumor chemotherapy drugs. In other words, the present disclosure is to provide a tumor chemotherapy sensitizer that enhances the antitumor effect of chemotherapy drugs. In the present disclosure, for the first time, the RIP2 inhibitor is found to enhance the antitumor activity of chemotherapy drugs; after combined administration, the ratios of CD45+ cells, neutrophils, dendritic cells, macrophages, CD4+ T cells, CD8+ T cells, and γ/δ T cells in the tumor microenvironment are up-regulated, and tumor chemotherapy resistance is reversed.

A first aspect of the present disclosure provides a use of a RIP2 inhibitor combined with a chemotherapy drug in the preparation of a medicament for treating and/or preventing a tumor.

Herein, the chemotherapy drug may be conventional in the art, preferably including one or more of the following, but not limited to: Paclitaxel, Docetaxel, Cisplatin, Oxaliplatin, Capecitabine, 5-Fluorouracil, Gemcitabine, Pemetrexed, Chidamide, and Bortezomib;

Herein, the RIP2 inhibitor preferably includes one or more of GSK583, WEHI-345, SB203580, OD36, OD38, and a derivative thereof.

The derivative of GSK583 is preferably GSK2983559.

The derivative may be a common type or an uncommon type of molecule in the art.

The tumor is preferably a tumor related to RIP2 signaling pathway, more preferably melanoma, lung cancer, breast cancer, gastric cancer, intestinal cancer, or pancreatic cancer.

A second aspect of the present disclosure provides a method for treating and/or preventing a tumor by jointly administering a RIP2 inhibitor and a chemotherapy drug to a patient in need thereof.

Herein, the RIP2 inhibitor is preferably administered in a dose of 1 mg/kg to 5 mg/kg.

The dose of the chemotherapy drug can be the conventional dose in the art, for example, the dose of PTX is 10 mg/kg, the dose of DTX is 5 mg/kg, the dose of GEM is 60 mg/kg, and the dose of OXA is 6 mg/kg.

The RIP2 inhibitor is preferably administered via intravenous injection, intramuscular injection, or oral administration.

In the method, the chemotherapy drug may be conventional in the art, preferably including one or more than one of the following, but not limited to: Paclitaxel, Docetaxel, Cisplatin, Oxaliplatin, Capecitabine, 5-Fluorouracil, Gemcitabine, Pemetrexed, Chidamide, and Bortezomib.

Herein, the RIP2 inhibitor preferably comprises one or more than one of GSK583, WEHI-345, SB203580, OD36, OD38, and a derivative thereof.

The derivative of GSK583 is preferably GSK2983559.

The derivative may be a common type or an uncommon type of molecule in the art.

In the method, the tumor is preferably a tumor related to the RIP2 signaling pathway, such as melanoma, lung cancer, breast cancer, gastric cancer, intestinal cancer, or pancreatic cancer.

A second aspect of the present disclosure provides a pharmaceutical composition comprising a RIP2 inhibitor and a chemotherapy drug, and the mass ratio of the RIP2 inhibitor to the chemotherapy drug is 1:(1 to 60).

In the pharmaceutical composition, the mass ratio is preferably 1:5, 1:1, 1:40, 1:6, 1:60, or 1:10.

Herein, the RIP2 inhibitor preferably comprises one or more of GSK583, WEHI-345, SB203580, OD36, OD38, and a derivative thereof.

The derivative of GSK583 is preferably GSK2983559.

The derivative may be a common type or an uncommon type of molecule in the art.

In the pharmaceutical composition, the chemotherapy drug may be conventional in the art, preferably including one or more than one of the following, but not limited to: Paclitaxel, Docetaxel, Cisplatin, Oxaliplatin, Capecitabine, 5-Fluorouracil, Gemcitabine, Pemetrexed, Chidamide, and Bortezomib.

In addition, the chemotherapy drug of the present disclosure may also include, but is not limited to:

Platinum: cDDP (Cisplatin), CBP (Carboplatin), L-OHP (Oxaliplatin), NDP (Nedaplatin), etc.

Microtubule inhibitors: PTX (Paclitaxel), TXT (Taxotere), VLB (Vinblastine), VCR (Vincristione), VNR (Vinorelbine), etc.

Antimetabolites: 5-Fu (5-Fluorouracil), XELOX (Capecitabine), 6-TG (Thioguanine), AG337 (Nolatrexed), FT-207 (Tegafur), HCFU (Carmofur), 5′-DFUR (Doxifluridine), UFT (Compund Tegafu), Ara-C(Cytarabine), etc.

Topoisomerase II inhibitors: VP16-213 (Etoposide), VM-26 (Teniposide), etc.

Cyclophosphamide, Pemetrexed, Chidamide, Bortezomib, etc.

Preferred are Paclitaxel, Docetaxel, 5-Fu, Oxaliplatin, cis-platinum, Capecitabine, Gemcitabine, Cyclophosphamide, Pemetrexed, Chidamide, and Bortezomib.

The RIP2 of the present disclosure is receptor-interacting protein 2.

The drug of the present disclosure is a RIP2 inhibitor including, but not limited to, GSK583, and the CAS number of the GSK583 is 1346547-00-9.

The present disclosure provides a pharmaceutical composition for treating a tumor, which has been confirmed in a series of in vivo experiments. In some embodiments of the present disclosure, the RIP2 inhibitor GSK583 is used in different doses according to different conditions of the tumor model in mice (tumor size, number of treatments, interval, etc.). The RIP2 inhibitor is administered at a typical dose of 1 mg/kg every 4 days for a total of 3 to 4 doses.

On the basis of common knowledge in the art, the above preferred conditions can be combined arbitrarily to obtain preferred examples of the present disclosure.

The reagent and raw material used in the present disclosure are all commercially available.

The positive and progressive effect of the present disclosure is:

For the first time, the RIP2 inhibitor is found to enhance the antitumor activity of chemotherapy drugs; after combined administration, the ratios of CD45+ cells, neutrophils, dendritic cells, macrophages, CD4+ T cells, CD8+ T cells, and γ/δ T cells in the tumor microenvironment are up-regulated, and tumor chemotherapy resistance is reversed.

### BRIEF DESCRIPTION OF THE DRAWINGS

FIG. 1 shows the establishment of a melanoma (B16F10) tumor-bearing model in C57BL/6 mice and the administration regimen of the RIP2 inhibitor GSK583 combined with Paclitaxel (PTX). Herein, A shows the establishment scheme of the melanoma tumor-bearing model in C57BL/6 mice; B shows the administration regimen of GSK583 combined with PTX for treating the melanoma tumor-bearing model in C57BL/6 mice.

FIG. 2 shows that GSK583 significantly enhances the inhibition of PTX on in vivo growth of melanoma in mice. A of FIG. 2 shows a graph of tumor volume growth change in different administration groups. B of FIG. 2 shows a graph of body weight change in tumor-bearing mice in different administration groups. C of FIG. 2 shows the results of tumor tissue weight in different administration groups. D of FIG. 2 shows the results of tumor weight inhibition rate in different administration groups.

FIG. 3 shows that GSK583 combined with PTX can increase the proportion of immune cells in tumor tissues of the melanoma tumor-bearing model in mice. A of FIG. 3 shows the proportion of CD45+ cells in tumor tissues in different administration groups. B of FIG. 3 shows the proportion of neutrophils in tumor tissues in different administration groups. C of FIG. 3 shows the proportion of dendritic cells in tumor tissues in different administration groups. D of FIG. 3 shows the proportion of macrophages in tumor tissues in different administration groups. E of FIG. 3 shows the proportion of CD4+ T cells in tumor tissues in different administration groups. F of FIG. 3 shows the proportion of CD8+ T cells in tumor tissues in different administration groups. G of FIG. 3 shows the proportion of γ/δ T cells in tumor tissues in different administration groups.

FIG. 4 shows the establishment of a melanoma (B16F10) tumor-bearing model in C57BL/6 mice and the administration regimen of the RIP2 inhibitor GSK583 combined with Docetaxel (DTX). A of FIG. 4 shows the establishment scheme of the melanoma model in C57BL/6 mice. B of FIG. 4 shows the administration regimen of GSK583 combined with DTX for treating melanoma in C57BL/6 mice.

FIG. 5 shows that GSK583 significantly enhances the inhibition of DTX on in vivo growth of melanoma in mice. A of FIG. 5 shows a graph of tumor volume growth change in different administration groups. B of FIG. 5 shows a graph of body weight change in tumor-bearing mice in different administration groups. C of FIG. 5 shows a photograph of tumor tissues in different administration groups at the end of the experiment. D of FIG. 5 shows the results of tumor weight inhibition rate in different administration groups.

FIG. 6 shows the establishment of a melanoma (B16F10) tumor-bearing model in C57BL/6 mice and the administration regimen of the RIP2 inhibitor GSK583 combined with Gemcitabine (GEM). A of FIG. 6 shows the establishment scheme of the melanoma model in C57BL/6 mice. B of FIG. 6 shows the administration regimen of GSK583 combined with GEM for treating the melanoma tumor-bearing model in C57BL/6 mice.

FIG. 7 shows that GSK583 significantly enhances the inhibition of GEM on in vivo growth of melanoma in C57BL/6 mice. A of FIG. 7 shows a graph of tumor volume growth change in different administration groups. B of FIG. 7 shows a photograph of tumor tissues in different administration groups at the end of the experiment. C of FIG. 7 shows the results of tumor tissue weight in different administration groups. D of FIG. 7 shows the results of tumor weight inhibition rate in different administration groups.

FIG. 8 shows the establishment of a colorectal cancer (MC38) tumor-bearing model in C57BL/6 mice and the administration regimen of the RIP2 inhibitor GSK583 combined with Gemcitabine (GEM). A of FIG. 8 shows the establishment scheme of the colorectal cancer tumor-bearing model in C57BL/6 mice. B of FIG. 8 shows the administration regimen of GSK583 combined with GEM for treating the colorectal cancer tumor-bearing model in C57BL/6 mice.

FIG. 9 shows that GSK583 significantly enhances the inhibition of GEM on in vivo growth of colorectal cancer in C57BL/6 mice. A of FIG. 9 shows a graph of tumor volume growth change in different administration groups. B of FIG. 9 shows a photograph of tumor tissues in different administration groups at the end of the experiment. C of FIG. 9 shows the results of tumor tissue weight in different administration groups. D of FIG. 9 shows the results of tumor weight inhibition rate in different administration groups.

FIG. 10 shows the establishment of a colorectal cancer (MC38) tumor-bearing model in C57BL/6 mice and the administration regimen of the RIP2 inhibitor GSK583 combined with Oxaliplatin (OXA). A of FIG. 10 shows the establishment scheme of the colorectal cancer tumor-bearing model in C57BL/6 mice. B of FIG. 10 shows the administration regimen of GSK583 combined with GEM for treating the colorectal cancer tumor-bearing model in C57BL/6 mice.

FIG. 11 shows that GSK583 significantly enhances the inhibition of OXA on in vivo growth of colorectal cancer in C57BL/6 mice. A of FIG. 11 shows a graph of tumor volume growth change in different administration groups. B of FIG. 11 shows a photograph of tumor tissues in different administration groups at the end of the experiment. C of FIG. 11 shows the results of tumor tissue weight in different administration groups. D of FIG. 11 shows the results of tumor weight inhibition rate in different administration groups.

### DETAILED DESCRIPTION OF THE PREFERRED EMBODIMENT

The examples of the present disclosure are described in detail below. The examples described below are exemplary and are only used to explain the present disclosure, and cannot be construed as limiting the present disclosure. The examples without indication of specific techniques or conditions shall be implemented in accordance with the techniques or conditions described in the literature in the art or in accordance with the product specification. The reagents or instruments used without indication of the manufacturer are all commercially available conventional products.

### Example 1: Significant Enhancement of Inhibition of PTX on In Vivo Growth of Melanoma in Mice by RIP2 Inhibitor GSK583

Both of the test substances PTX (Sigma) and GSK583 (MCE) were prepared as a 20× DMSO stock solution, which was diluted with normal saline containing 5% (v/v) polyoxyethylene castor oil (Cremophor EL) to the administration concentration before use.

Experimental animals and tumor strains: SPF grade male wild-type C57BL/6 mice, 6 to 8 weeks old, purchased from Beijing Vital River Laboratory Animal Technology Co., Ltd. Cells used in the experiment: murine melanoma cell line (B16-F10), purchased from ATCC.

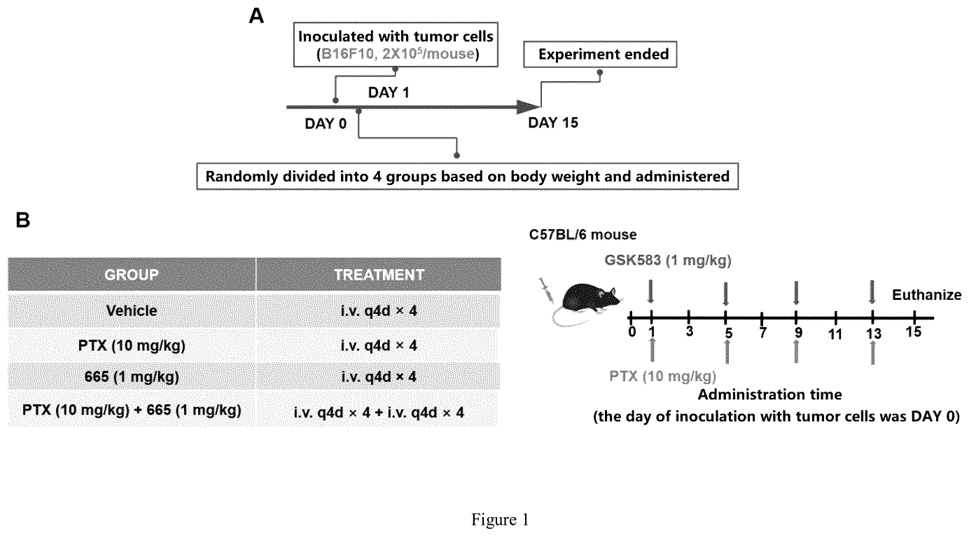

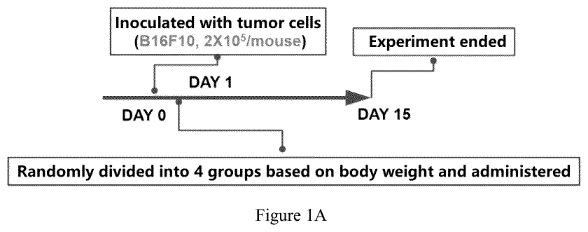

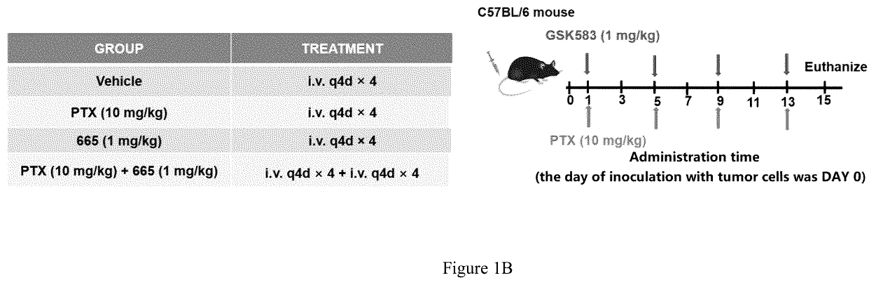

Mice were randomly divided into 4 groups with 8 mice in each group based on body weight according to the method shown in (A of FIG. 1 and B of FIG. 1), namely: (i) vehicle control group; (ii) GSK583 (1 mg/kg) group; (iii) PTX (10 mg/kg) group; (iv) GSK583 (1 mg/kg)+PTX (10 mg/kg) group. PTX, GSK583, and vehicle were administered intravenously once every 4 days for a total of 4 doses. The mice were subcutaneously inoculated with B16-F10 cells with a concentration of 2× 10⁶ cells/mL and 0.1 mL/mouse, and the day of inoculation was recorded as DAY 0. The administration was started on DAY 1. During the administration, the animals were weighed every 2 days. The long diameter and short diameter of the tumor were measured with a vernier caliper. The tumor size was calculated by the formula: (½)×long diameter×(short diameter) 2. The experiment was terminated on DAY 15. After the mice were euthanized and dissected, the tumor was weighed and the tumor inhibition rate was calculated.

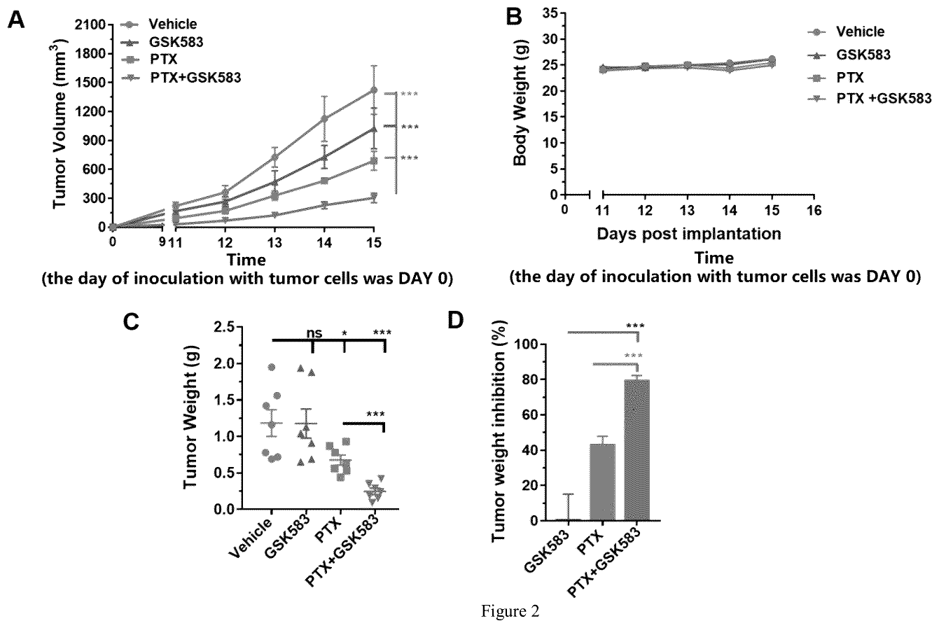

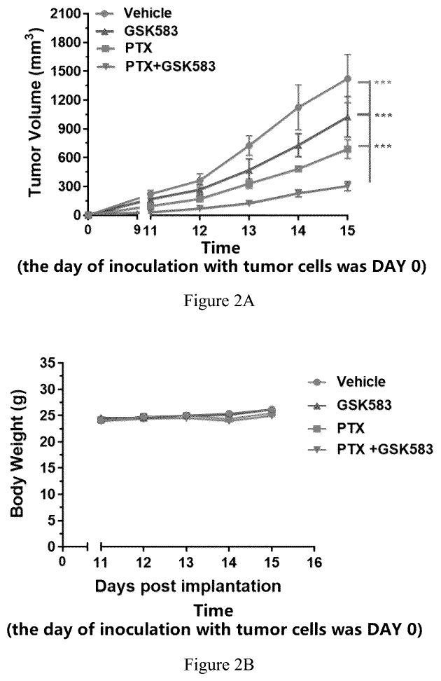

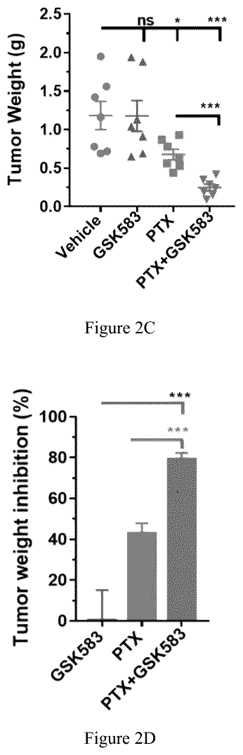

Experimental results (as shown in FIG. 2):

In the melanoma tumor-bearing model in C57BL/6 mice, both PTX and GSK583+PTX significantly inhibited tumor growth compared to the vehicle control group, and the growth rate of tumor volume was slower than that of the control group (A of FIG. 2). At the end of the administration, the tumor weight of animals in both groups was significantly and statistically lower than that of the control group (C of FIG. 2 and D of FIG. 2). The tumor inhibitory effect of GSK583+PTX was significantly stronger than that of PTX (C of FIG. 2 and D of FIG. 2). GSK583 alone had no significant inhibitory effect on tumor growth (A, C, and D of FIG. 2). No significant differences in body weight changes were observed between groups of mice (B of FIG. 2). It can be seen that the RIP2 inhibitor GSK583 significantly enhances the inhibition of PTX on in vivo growth of melanoma in mice.

### Example 2: Flow Cytometry Detection of Proportion of Immune Cells in Tumor Microenvironment of Melanoma Tumor-Bearing Mice Treated with GSK583 Combined with PTX

Preparation of enzyme lysis buffer: 1 g of collagenase type IV (Sigma C5138), 100 mg of hyaluronidase (Sigma H6254), and 20,000 units of DNase type IV (Sigma D5025) were added to 80 mL of HBSS and mixed well. The mixture was then added with HBSS to make up a volume of 100 mL to obtain a 100×enzyme lysis buffer, which was filtered through a 0.22 μm filter membrane and stored at −20° C., and diluted with HBSS to 1× concentration before use.

Dissociation of tumor tissues: Tumor tissues were removed and weighed (0.04 to 1 g), then cut into small pieces of 2×2 mm with a scalpel, added with 2.5 mL of enzyme lysis mixture, and transferred to a gentleMACS C Tube (Miltenyi Biotec 130-096-334). The C Tube was inverted on a gentleMACS Dissociator (Miltenyi Biotec 130-093-235), and the program “gentleMACS Program m_impTumor_02” was run. The C Tube was removed from the gentleMACS Dissociator and incubated at 37° C. for 40 minutes. The C Tube was inverted on the gentleMACS Dissociator again, and the program “gentleMACS Program m_impTumor_03” was run.

The mixture was filtered through a 70 μm filter membrane (BD Biosciences, Cat: 352235), centrifuged at 300×g for 7 minutes, and the supernatant was discarded. 5 mL of red blood cell lysis buffer (Biolegend, Cat: 420301) was added thereto, then the mixture was lysed at room temperature for 5 minutes, and 10 mL of phosphate-buffered saline (PBS; EallBio Life Science, Cat: 03.15018C) was added thereto. The mixture was centrifuged at 1,000 rpm for 5 minutes at 4° C., and the supernatant was discarded. Cell staining buffer (Biolegend, Cat: 420201) was added and mixed well.

Flow cytometry detection: After counting the dissociated tumor tissues, the cell concentration was adjusted, and 200 μL/well of cells (5 to 10×10⁵ cells/well) were added to a 96 U-bottom plate. The plate was centrifuged at 350×g for 5 minutes, and the supernatant was discarded. 100 μL/well of 1×blocking buffer (cell staining buffer containing 1:200 diluted CD16/CD32) was added thereto, and the cells were incubated in the dark at 4° C. for 15 to 20 minutes. The plate was centrifuged at 350×g for 5 minutes, and the supernatant was discarded. 100 μL/well of prepared antibody mixture was added thereto, and the cells were incubated in the dark at 4° C. for 30 minutes. The plate was centrifuged at 350×g for 5 minutes, and the supernatant was discarded. 100 μL/well of cell staining buffer was then added thereto for washing. The plate was centrifuged at 350×g for 5 minutes, and the supernatant was discarded. The washing was repeated twice. After the last washing, the cells were resuspended in 100 μL/well of 4% paraformaldehyde (Beyotime, P0099) and incubated overnight at 4° C. The plate was centrifuged at 350×g for 5 minutes, and the supernatant was discarded. The cells were resuspended in 100 μL/well of cell staining buffer, and then detected by flow cytometry.

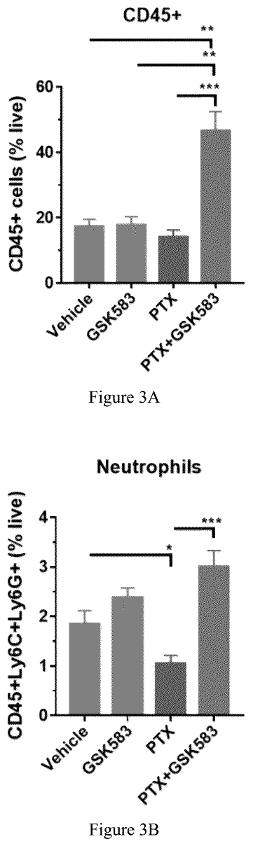

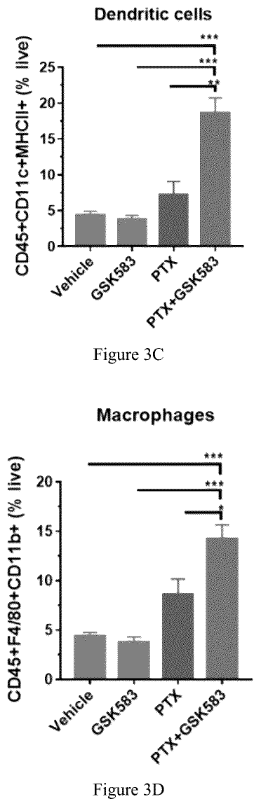

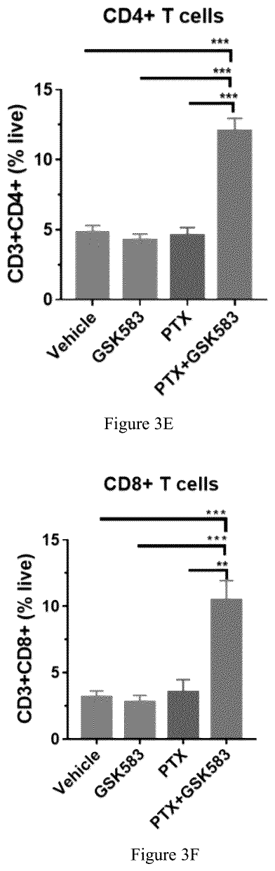

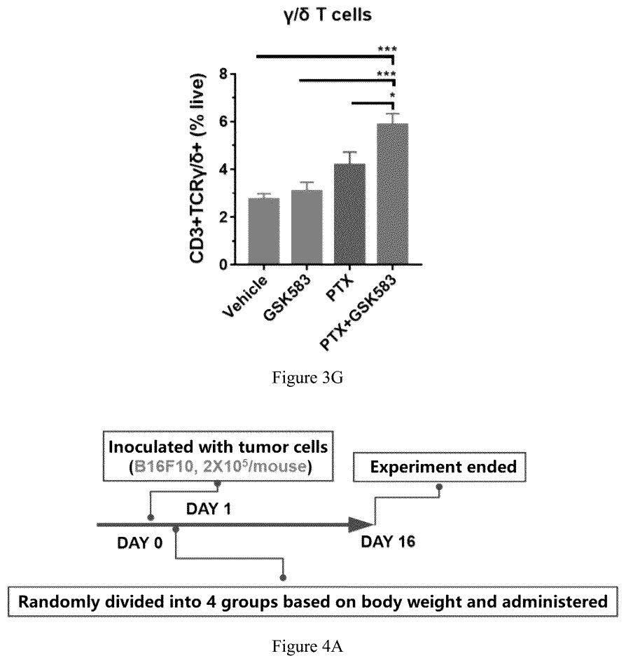

Experimental results (as shown in FIG. 3):

The GSK583+PTX combined administration group showed a significant increase in the proportion of CD45+ cells, neutrophils, dendritic cells, macrophages, CD4+ T cells, CD8+ T cells, and γ/δ T cells compared to the vehicle control group, GSK583-alone administration group, and PTX-alone administration group (A to G of FIG. 3). The PTX-alone administration group showed a significant decrease in the proportion of neutrophils compared to the vehicle control group (B of FIG. 3), which is consistent with the clinically reported decrease in the number of neutrophils in tumor patients treated with PTX. Thus, it can be inferred that the combined administration of GSK583+PTX favors an increase in the proportion of CD45+ cells, neutrophils, dendritic cells, macrophages, CD4+ T cells, CD8+ T cells, and γ/δ T cells in murine tumor tissues, exerting an inhibitory effect on tumor growth.

### Example 3: Significant Enhancement of Inhibition of DTX on In Vivo Growth of Melanoma in C57BL/6 Mice by RIP2 Inhibitor GSK583

Both of the test substances DTX and GSK583 were prepared as a 20×DMSO stock solution, which was diluted with normal saline containing 5% polyoxyethylene castor oil (Cremophor EL) to the administration concentration before use.

Experimental animals and tumor strains: SPF grade male wild-type C57BL/6 mice, 6 to 8 weeks old, purchased from Beijing Vital River Laboratory Animal Technology Co., Ltd. Cells used in the experiment: murine melanoma cells (B16-F10), purchased from ATCC.

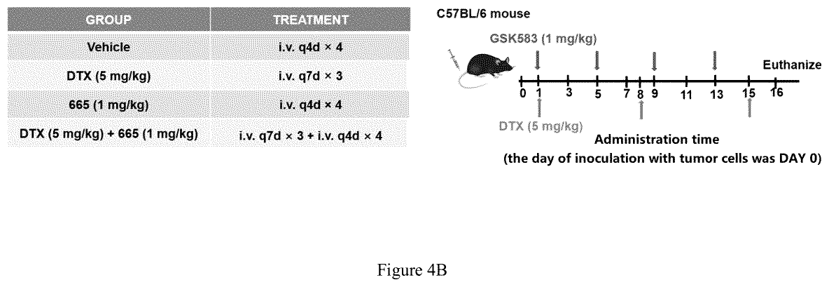

Mice were randomly divided into 4 groups with 8 mice in each group based on body weight according to the method shown in (A and B of FIG. 4), namely: (i) vehicle control group; (ii) GSK583 (1 mg/kg) group; (iii) Docetaxel (DTX; 5 mg/kg) group; (iv) GSK583 (1 mg/kg)+DTX (5 mg/kg) group. DTX was administered intravenously once every 7 days for a total of 3 doses; GSK583 and vehicle control were administered intravenously once every 4 days for a total of 4 doses. The mice were subcutaneously inoculated with B16-F10 cells with a concentration of 2× 10⁶ cells/mL and 0.1 mL/mouse, and the day of inoculation was recorded as DAY 0. The administration was started on DAY 1. During the administration, the animals were weighed every 2 days. The long diameter and short diameter of the tumor were measured with a vernier caliper. The tumor size was calculated by the formula: (½)×long diameter×(short diameter) 2. The mice were euthanized on DAY 16, and after dissection, the tumor was weighed and the tumor inhibition rate was calculated.

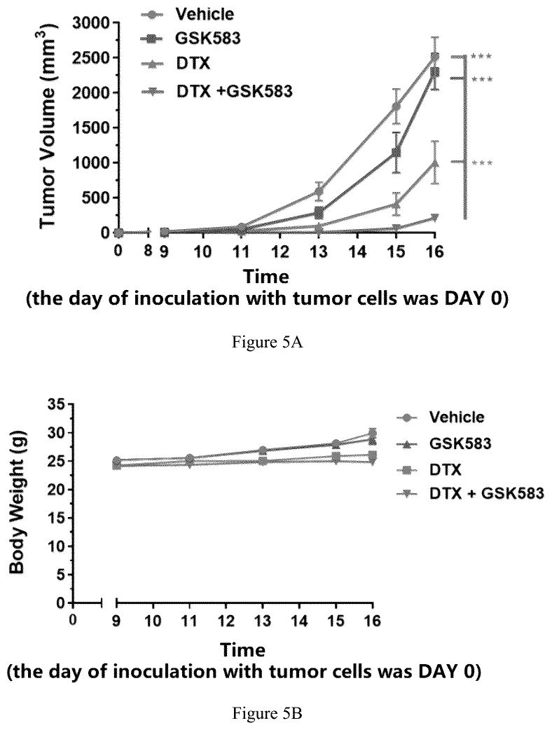

Experimental results (as shown in FIG. 5):

In the melanoma (B16F10) tumor-bearing model in C57BL/6 mice, both DTX and GSK583+DTX significantly inhibited tumor growth compared to the vehicle control group, and the growth rate of tumor volume was slower than that of the control group (A of FIG. 5). At the end of the administration, the tumor weight of animals in both DTX and GSK583+DTX groups was significantly and statistically lower than that of the control group (C of FIG. 5 and D of FIG. 5). The tumor inhibitory effect of GSK583+DTX was significantly stronger than that of the DTX-alone group (C of FIG. 5 and D of FIG. 5). GSK583 alone had no significant inhibitory effect on tumor growth (A, C, and D of FIG. 5). No significant differences in body weight changes were observed between groups of mice (B of FIG. 5). It can be seen that the RIP2 inhibitor GSK583 significantly enhances the inhibition of DTX on in vivo growth of melanoma in mice.

### Example 4: Significant Enhancement of Inhibition of Gemcitabine (GEM) on In Vivo Growth of Melanoma in Mice by RIP2 Inhibitor GSK583

The test substance GEM was prepared in normal saline, and GSK583 was prepared as a 20×DMSO stock solution, which was diluted with normal saline containing 5% polyoxyethylene castor oil (Cremophor EL) to the administration concentration before use.

Experimental animals and tumor strains: SPF grade male wild-type C57BL/6 mice, 6 to 8 weeks old, purchased from Beijing Vital River Laboratory Animal Technology Co., Ltd. Cells used in the experiment: murine melanoma cells (B16-F10), purchased from ATCC.

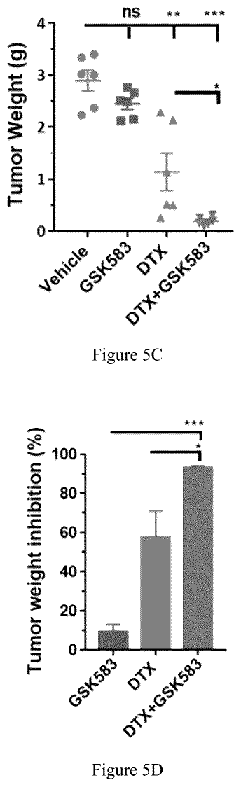

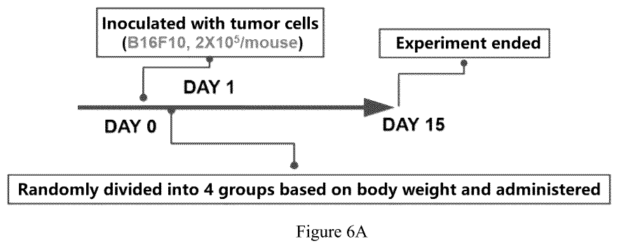

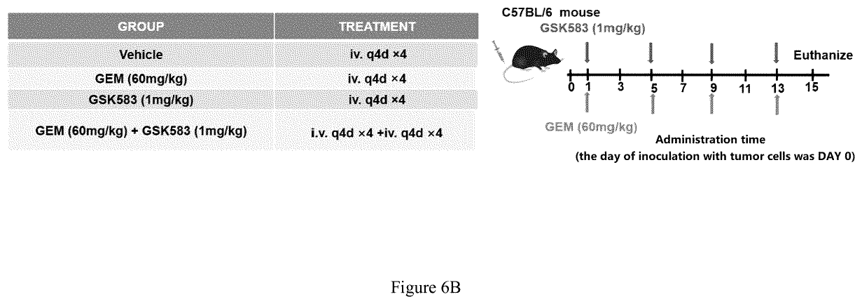

Mice were randomly divided into 4 groups with 8 mice in each group based on body weight according to the method shown in (A and B of FIG. 6), namely: (i) vehicle control group; (ii) GSK583 (1 mg/kg) group; (iii) GEM (60 mg/kg) group; (iv) GSK583 (1 mg/kg)+GEM (60 mg/kg) group. GEM, GSK583, and vehicle control were administered intravenously once every 4 days for a total of 4 doses. The mice were subcutaneously inoculated with B16-F10 cells with a concentration of 2× 10⁶ cells/mL and 0.1 mL/mouse, and the day of inoculation was recorded as DAY 0. The administration was started on DAY 1. During the administration, the animals were weighed every 2 days. The long diameter and short diameter of the tumor were measured with a vernier caliper. The tumor size was calculated by the formula: (½)× long diameter×(short diameter) 2. The experiment was terminated on DAY 15. After the mice were euthanized and dissected, the tumor was weighed and photographed, and the tumor inhibition rate was calculated.

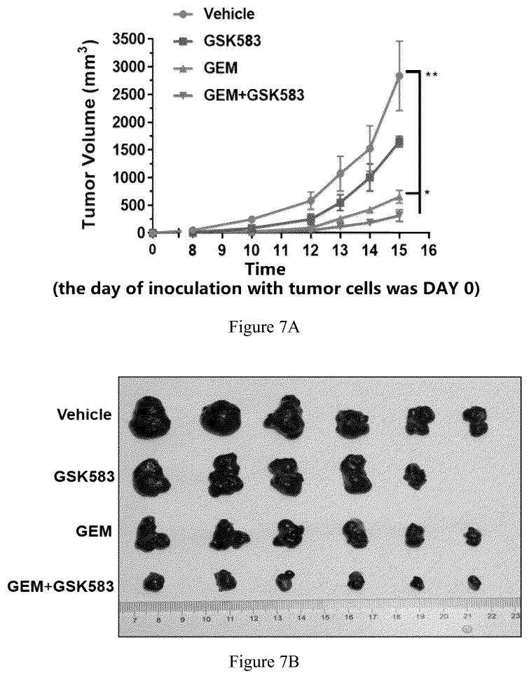

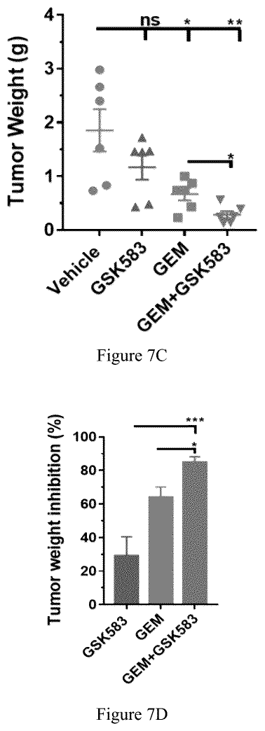

Experimental results (as shown in FIG. 7):

In the melanoma (B16F10) tumor-bearing model in C57BL/6 mice, both GEM and GSK583+GEM significantly inhibited tumor growth compared to the vehicle control group, and the growth rate of tumor volume was slower than that of the control group (A of FIG. 7). At the end of the administration, the tumor weight of animals in both groups was significantly and statistically lower than that of the control group (B, C, and D of FIG. 7). The tumor inhibitory effect of GSK583+GEM was significantly stronger than that of the GEM-alone group (C and D of FIG. 7). GSK583 alone had no significant inhibitory effect on tumor growth (A, C, and D of FIG. 7). It can be seen that the RIP2 inhibitor GSK583 significantly enhances the inhibition of GEM on in vivo growth of melanoma in mice.

### Example 5: Significant Enhancement of Inhibition of Gemcitabine (GEM) on In Vivo Growth of Colorectal Cancer (MC38) in C57BL/6 Mice by RIP2 Inhibitor GSK583

The test substance GEM was prepared in normal saline, and GSK583 was prepared as a 20×DMSO stock solution, which was diluted with normal saline containing 5% polyoxyethylene castor oil (Cremophor EL) to the administration concentration before use.

Experimental animals and tumor strains: SPF grade male wild-type C57BL/6 mice, 6 to 8 weeks old, purchased from Beijing Vital River Laboratory Animal Technology Co., Ltd. Cells used in the experiment: murine colorectal cancer cells (MC38), purchased from Union Cell Bank (China).

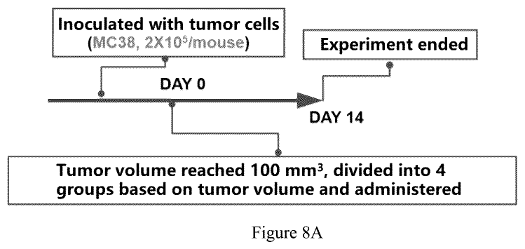

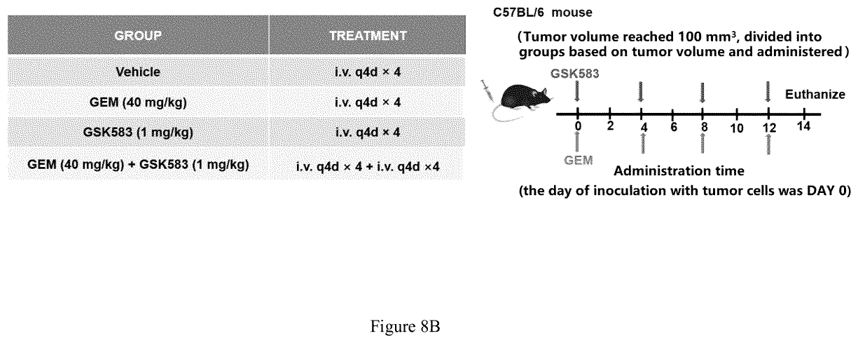

According to the method shown in (A and B of FIG. 8), the mice were subcutaneously inoculated with MC38 cells with a concentration of 2×10⁶ cells/mL and 0.1 mL/mouse. When the tumor grew to a volume within the range of 50 to 100 mm³, the mice were randomly divided into 4 groups with 8 mice in each group based on tumor volume, and the administration was started with the following regimen: (i) vehicle control group; (ii) GSK583 (1 mg/kg) group; (iii) GEM (40 mg/kg) group; (iv) GSK583 (1 mg/kg)+GEM (40 mg/kg) group. GEM, GSK583, and vehicle were administered intravenously once every 4 days for a total of 4 doses. The day of grouping and administration was recorded as DAY 0. During the administration, the animals were weighed every 3 days. The long diameter and short diameter of the tumor were measured with a vernier caliper. The tumor size was calculated by the formula: (½)× long diameter× (short diameter) 2. The experiment was terminated on DAY 14. After the mice were euthanized and dissected, the tumor was weighed and photographed, and the tumor inhibition rate was calculated.

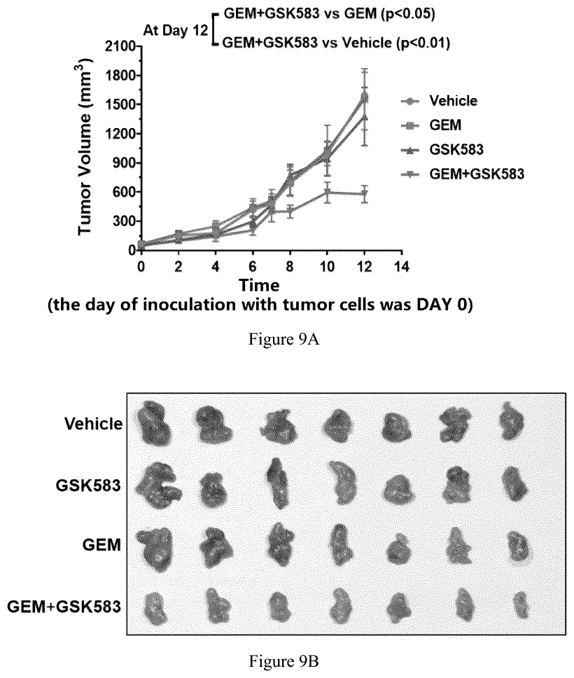

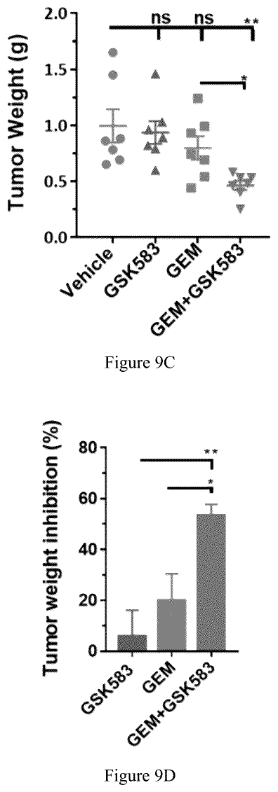

Experimental results (as shown in FIG. 9):

In the colorectal cancer (MC38) tumor-bearing model in C57BL/6 mice, GEM (40 mg/kg) had no significant inhibitory effect on tumor growth and the growth rate of tumor volume compared to the vehicle control group, but GSK583+GEM significantly inhibited tumor growth, and the growth rate of tumor volume was slower than that of the control group and the GEM group (A of FIG. 9). At the end of the administration, the tumor weight of animals in the GSK583+GEM group was significantly and statistically lower than that of the control group (B, C, and D of FIG. 9). The tumor inhibitory effect of GSK583+GEM was significantly stronger than that of the GEM-alone group (C and D of FIG. 9). GSK583 alone had no significant inhibitory effect on tumor growth (A, C, and D of FIG. 9), which indicated that the tumor inhibitory effect of GSK583+GEM was significantly stronger than that of the GEM-alone group. It can be seen that the RIP2 inhibitor GSK583 significantly enhances the inhibition of GEM on in vivo growth of colorectal cancer in mice.

### Example 6: Significant Enhancement of Inhibition of Oxaliplatin (OXA) on In Vivo Growth of Colorectal Cancer (MC38) in C57BL/6 Mice by RIP2 Inhibitor GSK583

The test substance OXA was prepared in 5% dextrose solution, and GSK583 was prepared as a 20×DMSO stock solution, which was diluted with normal saline containing 5% polyoxyethylene castor oil (Cremophor EL) to the administration concentration before use.

Experimental animals and tumor strains: SPF grade male wild-type C57BL/6 mice, 6 to 8 weeks old, purchased from Beijing Vital River Laboratory Animal Technology Co., Ltd. Cells used in the experiment: murine colorectal cancer cells (MC38), purchased from Union Cell Bank (China).

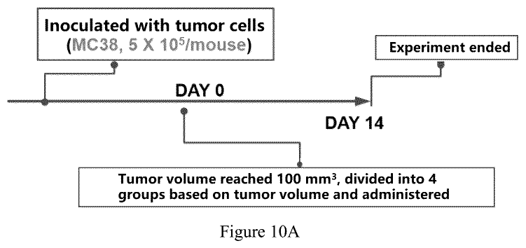

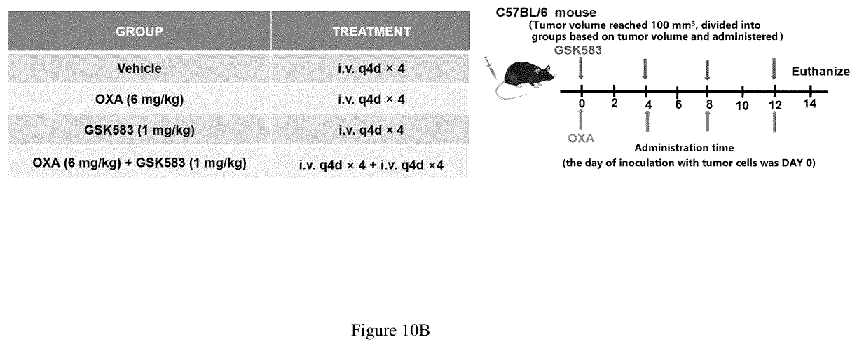

According to the method shown in (A and B of FIG. 10), the mice were subcutaneously inoculated with MC38 cells with a concentration of 5×10⁶ cells/mL and 0.1 mL/mouse. When the tumor grew to a volume of 100 mm³, the mice were randomly divided into 4 groups with 8 mice in each group based on tumor volume, and the administration was started with the following regimen: (i) vehicle control group; (ii) GSK583 (1 mg/kg) group; (iii) OXA (6 mg/kg) group; (iv) GSK583 (1 mg/kg)+OXA (6 mg/kg) group. OXA, GSK583, and vehicle were administered intravenously once every 4 days for a total of 4 doses. The day of grouping and administration was recorded as DAY 0. During the administration, the animals were weighed every 3 days. The long diameter and short diameter of the tumor were measured with a vernier caliper. The tumor size was calculated by the formula: (½)×long diameter×(short diameter) 2. The experiment was terminated on DAY 14. After the mice were euthanized and dissected, the tumor was weighed and photographed, and the tumor inhibition rate was calculated.

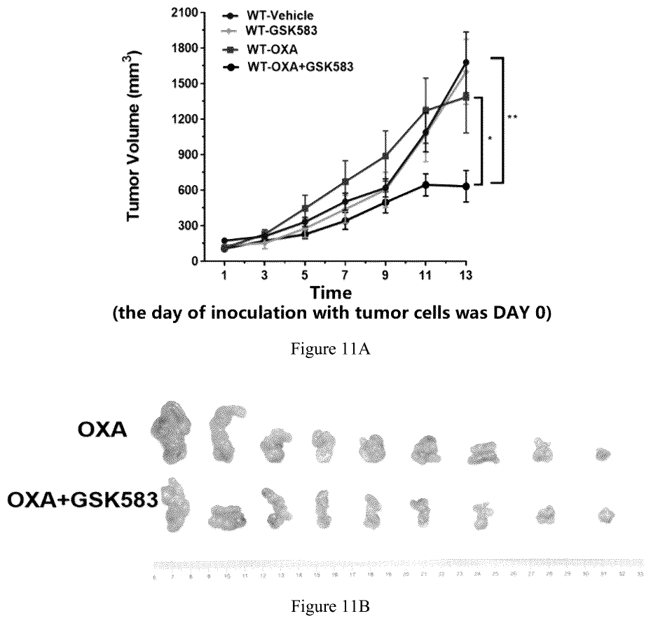

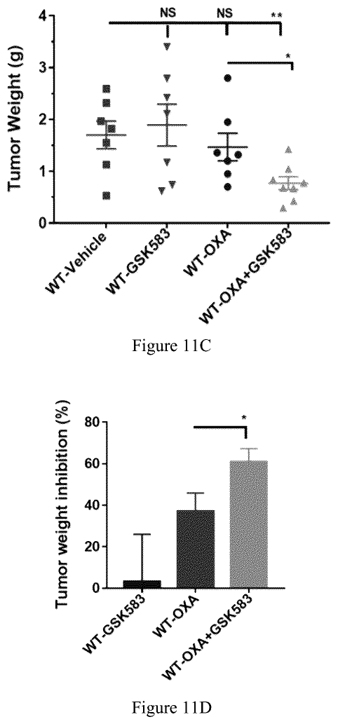

Experimental results (as shown in FIG. 11):

In the colorectal cancer cell (MC38) tumor-bearing model in C57BL/6 mice, OXA (6 mg/kg) had no significant inhibitory effect on tumor growth and the growth rate of tumor volume compared to the vehicle control group, but GSK583+OXA significantly inhibited tumor growth, and the growth rate of tumor volume was slower than that of the control group and the OXA group (A of FIG. 11). At the end of the administration, the tumor weight of animals in the GSK583+OXA group was significantly and statistically lower than that of the control group and the OXA group (B, C, and D of FIG. 11), which indicated that the tumor inhibitory effect of GSK583+OXA was significantly stronger than that of the OXA-alone group. GSK583 alone had no significant inhibitory effect on tumor growth (A, C, and D of FIG. 11). It can be seen that the RIP2 inhibitor GSK583 significantly enhances the inhibition of OXA on in vivo growth of colorectal cancer in mice.

Those skilled in the art may combine and incorporate the different embodiments or examples described in this specification and their features without conflicting with each other.

Although the examples of the present disclosure are illustrated and described above, it can be understood that the above examples are illustrative and should not be construed as limiting the present disclosure. Those skilled in the art can make changes, modifications, substitutions, and variations based on the above examples within the scope of the present disclosure.

1-3. (canceled)

4. A method for treating or preventing a tumor,
comprising administering a RIP2 inhibitor combined with a chemotherapy drug to a patient in need thereof.

5. The method according to claim 4, wherein the RIP2 inhibitor is administered in a dose of 1 mg/kg to 5 mg/kg;
or, the RIP2 inhibitor is administered via intravenous injection, intramuscular injection, or oral administration.

6. The method according to claim 4, wherein the chemotherapy drug comprises one or more of the following: Paclitaxel, Docetaxel, Cisplatin, Oxaliplatin, Capecitabine, 5-Fluorouracil, Gemcitabine, Pemetrexed, Chidamide, and Bortezomib;
or, the RIP2 inhibitor comprises one or more of GSK583, WEHI-345, OD36, OD38.

7. The method according to claim 4, wherein the tumor is a RIP2 related tumor.

8. A pharmaceutical composition, comprising a RIP2 inhibitor and a chemotherapy drug, and the mass ratio of the RIP2 inhibitor to the chemotherapy drug is 1:(1 to 60).

9. The pharmaceutical composition according to claim 8, wherein the mass ratio is 1:5, 1:1, 1:40, 1:60, 1:6, or 1:10;
or, the RIP2 inhibitor comprises one or more of GSK583, WEHI-345, OD36, OD38, and a derivative thereof.

10. The pharmaceutical composition according to claim 8, wherein the chemotherapy drug comprises one or more of the following: Paclitaxel, Docetaxel, Cisplatin, Oxaliplatin, Capecitabine, 5-Fluorouracil, Gemcitabine, Pemetrexed, Chidamide, and Bortezomib.

11. The pharmaceutical composition according to claim 10, wherein the chemotherapy drug comprises one or more of the following: Paclitaxel, Docetaxel, Oxaliplatin, and Gemcitabine.

12. The pharmaceutical composition according to claim 9, wherein the RIP2 inhibitor is a derivative of GSK583.

13. The pharmaceutical composition according to claim 12, wherein the derivative of GSK583 is GSK2983559.

14. The method according to claim 6, wherein the chemotherapy drug comprises one or more of the following: Paclitaxel, Docetaxel, Oxaliplatin, and Gemcitabine.

15. The method according to claim 6, wherein the RIP2 inhibitor is a derivative of GSK583.

16. The method according to claim 15, wherein the derivative of GSK583 is GSK2983559.

17. The method according to claim 7, wherein the RIP2 related tumor is melanoma, lung cancer, breast cancer, intestinal cancer, gastric cancer, or pancreatic cancer.

18. The method according to claim 17, wherein the RIP2 related tumor is melanoma or intestinal cancer.
